(19) United States
(12) Patent Application Publication
(10) Pub. No.: US 2023/0128303 A1
(43) Pub. Date: Apr. 27, 2023

(54) Compact Robotic Endoscope

(71) Applicant: MicronVision Corp., Bellevue, WA (US)

(72) Inventor: Xiaolong OUYANG, Bellevue, WA (US)

(21) Appl. No.: 18/083,209

(22) Filed: Dec. 16, 2022

Related U.S. Application Data
(63) Continuation-in-part of application No. 17/941,884, filed on Sep. 9, 2022.
Application No. 17/941,884 is a continuation-in-part of application No. 17/843,217, filed on Jun. 17, 2022.
Application No. 17/843,217 is a continuation-in-part of application No. 17/349,674, filed on Jun. 16, 2021.
Application No. 17/349,674 is a continuation-in-part of application No. 63/417,340, filed on Oct. 19, 2022.
Application No. 17/941,884 is a continuation-in-part of application No. 17/745,526, filed on May 16, 2022.
Application No. 17/745,526 is a continuation-in-part of application No. 17/720,143, filed on Apr. 13, 2022.
Application No. 17/720,143 is a continuation-in-part of application No. 17/521,397, filed on Nov. 8, 2021.
Application No. 17/521,397 is a continuation-in-part of application No. 17/473,587, filed on Sep. 13, 2021, now Pat. No. 11,330,973.
Application No. 17/473,587 is a continuation-in-part of application No. 17/362,043, filed on Jun. 29, 2021, now Pat. No. 11,350,816.
Application No. 17/362,043 is a continuation-in-part of application No. 16/363,209, filed on Mar. 25, 2019.
Application No. 17/745,526 is a continuation-in-part of application No. 17/473,587, filed on Sep. 13, 2021, now Pat. No. 11,330,973.
Application No. 17/720,143 is a continuation-in-part of application No. 17/521,397, filed on Nov. 8, 2021.
Application No. 17/473,587 is a continuation-in-part of application No. 17/362,043, filed on Jun. 29, 2021, now Pat. No. 11,350,816.
Application No. 17/362,043 is a continuation-in-part of application No. PCT/US19/36060, filed on Jun. 7, 2019.
Application No. PCT/US19/36060 is a continuation-in-part of application No. 16/363,209, filed on Mar. 25, 2019.
Application No. PCT/US19/36060 is a continuation-in-part of application No. 16/363,209, filed on Mar. 25, 2019.
Application No. 16/363,209 is a continuation-in-part of application No. PCT/US17/53171, filed on Sep. 25, 2017.
Application No. 17/349,674 is a continuation-in-part of application No. 16/664,082, filed on Dec. 5, 2019, now Pat. No. 11,071,442.
(60) Provisional application No. 63/256,634, filed on Oct. 18, 2021.
(60) Provisional application No. 63/282,108, filed on Nov. 22, 2021.
(60) Provisional application No. 63/283,367, filed on Nov. 26, 2021.
(60) Provisional application No. 63/332,233, filed on Apr. 18, 2022.
(60) Provisional application No. 63/218,362, filed on Jul. 4, 2021.
(60) Provisional application No. 63/213,499, filed on Jun. 22, 2021.
(60) Provisional application No. 63/210,034, filed on Jun. 13, 2021.
(60) Provisional application No. 63/197,639, filed on Jun. 7, 2021.
(60) Provisional application No. 63/197,611, filed on Jun. 7, 2021.
(60) Provisional application No. 63/112,739, filed on Nov. 12, 2020.
(60) Provisional application No. 63/113,960, filed on Nov. 15, 2020.
(60) Provisional application No. 63/118,617, filed on Nov. 25, 2020.
(60) Provisional application No. 63/128,105, filed on Dec. 20, 2020.
(60) Provisional application No. 63/138,528, filed on Jan. 18, 2021.
(60) Provisional application No. 63/218,362, filed on Jul. 4, 2021.
(60) Provisional application No. 63/213,499, filed on Jun. 22, 2021.
(60) Provisional application No. 63/197,639, filed on Jun. 7, 2021.
(60) Provisional application No. 63/197,611, filed on Jun. 7, 2021.
(60) Provisional application No. 63/183,151, filed on May 3, 2021.
(60) Provisional application No. 63/153,252, filed on Feb. 24, 2021.
(60) Provisional application No. 63/149,338, filed on Feb. 14, 2021.
(60) Provisional application No. 63/138,751, filed on Jan. 18, 2021.
(60) Provisional application No. 63/129,703, filed on Dec. 23, 2020.
(60) Provisional application No. 63/124,803, filed on Dec. 13, 2020.
(60) Provisional application No. 63/121,924, filed on Dec. 6, 2020.
(60) Provisional application No. 63/121,246, filed on Dec. 4, 2020.
(60) Provisional application No. 63/107,344, filed on Oct. 29, 2020.
(60) Provisional application No. 63/087,935, filed on Oct. 6, 2020.
(60) Provisional application No. 63/083,932, filed on Sep. 27, 2020.
(60) Provisional application No. 63/077,675, filed on Sep. 13, 2020.
(60) Provisional application No. 63/077,635, filed on Sep. 13, 2020.
(60) Provisional application No. 63/183,151, filed on May 3, 2021.
(60) Provisional application No. 63/153,252, filed on Feb. 24, 2021.
(60) Provisional application No. 63/149,338, filed on Feb. 14, 2021.
(60) Provisional application No. 63/138,751, filed on Jan. 18, 2021.
(60) Provisional application No. 63/129,703, filed on Dec. 23, 2020.
(60) Provisional application No. 63/124,803, filed on Dec. 13, 2020.
(60) Provisional application No. 63/121,924, filed on Dec. 6, 2020.
(60) Provisional application No. 63/121,246, filed on Dec. 4, 2020.
(60) Provisional application No. 63/107,344, filed on Oct. 29, 2020.
(60) Provisional application No. 63/087,935, filed on Oct. 6, 2020.
(60) Provisional application No. 63/083,932, filed on Sep. 27, 2020.
(60) Provisional application No. 63/077,675, filed on Sep. 13, 2020.
(60) Provisional application No. 63/077,635, filed on Sep. 13, 2020.
(60) Provisional application No. 62/686,680, filed on Jun. 19, 2018.
(60) Provisional application No. 62/686,682, filed on Jun. 19, 2018.
(60) Provisional application No. 62/688,397, filed on Jun. 22, 2018.
(60) Provisional application No. 62/682,854, filed on Jun. 9, 2018.
(60) Provisional application No. 62/681,824, filed on Jun. 7, 2018.
(60) Provisional application No. 62/681,687, filed on Jun. 7, 2018.
(60) Provisional application No. 62/816,366, filed on Mar. 11, 2019.
(60) Provisional application No. 62/647,817, filed on Mar. 25, 2018.
(60) Provisional application No. 62/654,295, filed on Apr. 6, 2018.
(60) Provisional application No. 62/671,445, filed on May 15, 2018.
(60) Provisional application No. 62/816,366, filed on Mar. 11, 2019.
(60) Provisional application No. 62/842,297, filed on May 2, 2019.
(60) Provisional application No. 62/825,948, filed on Mar. 29, 2019.
(60) Provisional application No. 62/821,536, filed on Mar. 21, 2019.
(60) Provisional application No. 62/901,393, filed on Sep. 17, 2019.
(60) Provisional application No. 62/897,352, filed on Sep. 8, 2019.
(60) Provisional application No. 62/884,688, filed on Aug. 9, 2019.
(60) Provisional application No. 62/880,677, filed on Jul. 31, 2019.
(60) Provisional application No. 62/873,861, filed on Jul. 13, 2019.
(60) Provisional application No. 62/870,748, filed on Jul. 4, 2019.
(60) Provisional application No. 62/878,384, filed on Jul. 25, 2019.
(60) Provisional application No. 62/821,430, filed on Mar. 20, 2019.
(60) Provisional application No. 62/797,235, filed on Jan. 26, 2019.
(60) Provisional application No. 62/796,346, filed on Jan. 24, 2019.
(60) Provisional application No. 62/795,042, filed on Jan. 22, 2019.
(60) Provisional application No. 62/791,045, filed on Jan. 11, 2019.

(51) Int. Cl. A61B 5/00; A61B 1/307; A61B 1/06; A61B 1/04; A61B 1/045; A61B 1/00; A61M 13/00; A61B 1/05; A61B 1/12; G02B 21/16; A61B 1/015

(52) U.S. Cl. CPC A61B 5/0071; A61B 1/307; A61B 1/0638; A61B 1/043; A61B 1/045; A61B 1/00186; A61M 13/003; A61B 1/05; A61B 1/051; A61B 1/00096; A61B 1/0607; A61B 1/126; G02B 21/16; A61B 1/00052; A61B 1/00009; A61B 1/015; A61B 1/0655; A61B 2562/0233; A61M 2210/1085

(57) ABSTRACT

A medical endoscope that has a reusable portion to which either of two different single-use portions can be snapped in by hand to thereby form two different assembled endoscopes. When one of the single-use portions is assembled with the reusable portion, a motor in the reusable portion robotically rotates a cannula about a proximal end of the single use portion. When the other single-use portion is assembled with the reusable portion, the motor robotically angulates the distal end of the cannula. Another medical endoscope is single-use in its entirety and has motor-driven angulation of the cannula's distal end. Another has manually controlled angulation.

### REFERENCE TO RELATED APPLICATIONS

This application is a continuation in-part of each of Ser. Nos. 17/941,884, 17/843,217, and 17/349,674 and claims priority to Provisional Patent Application No. 63/417,340 filed Oct. 19, 2022

Ser. No. 17/941,884 is a continuation in-part of each of Ser. Nos. 17/745,526, 17/720,143, 17/521,397, 17/473,587 (now U.S. Pat. No. 11,330,973), Ser. Nos. 17/362,043, and 16/363,209 and claims priority to 4 provisional applications.

Ser. No. 17/745,526 is a continuation in-part of Ser. No. 17/473,587 and claims priority to 5 provisional applications.

Ser. No. 17/720,143 is a continuation in-part of Ser. No. 17/521,397.

Ser. No. 17/521,397 claims priority to 5 provisional applications.

Ser. No. 17/473,587 is a continuation in-part of each of Ser. No. 17/362,043 (now U.S. Pat. No. 11,350,816), PCT/US19/36060, and Ser. No. 16/363,209 and claims priority to 17 provisional applications.

Ser. No. 17/362,043 (now U.S. Pat. No. 11,350,816) claims priority to 13 provisional applications.

PCT/US19/36060 is a continuation in-part of Ser. No. 16/363,209 and claims priority to 7 provisional applications.

Ser. No. 16/363,209 is a continuation of PCT/US17/53171 and claims priority to 4 provisional applications.

PCT/US17/53171 claims priority to 15 provisional applications.

Ser. No. 17/843,217 is a division of Ser. No. 16/363,209.

Ser. No. 17/349,674 is a continuation in-part of Ser. No. 16/664,082.

Ser. No. 16/664,082 (now U.S. Pat. No. 11,071,442) claims priority to 26 provisional applications.

This application incorporates by reference the entirety of the foregoing patents and patent applications and claims the benefit of the filing date of each of the above-identified patent applications, as well as of the applications that they incorporated by reference, directly or indirectly, and the benefit of which they claim, including U.S. provisional applications, U.S. non-provisional applications, and international applications.

This patent application incorporates by reference each of the following U.S. patents and U.S. and international (PCT) patent applications:

Ser. No. 16/972,989 filed Dec. 7, 2020;

PCT/US21/50095 filed Sep. 13, 2021;

Ser. No. 17/835,624 filed Jun. 8, 2022;

PCT/US16/18670 filed Feb. 19, 2016;

Ser. No. 14/913,867 filed Feb. 23, 2016, now U.S. Pat. No. 10,874,287;

PCT/US16/65396 filed Dec. 7, 2016;

Ser. No. 15/371,858 filed Feb. 20, 2018, now U.S. Pat. No. 9,895,048;

Ser. No. 15/462,331 filed Mar. 17, 2017, now U.S. Pat. No. 10,524,636;

Ser. No. 15/651,526 filed Jul. 17, 2017, now U.S. Pat. No. 10,278,563;

Ser. No. 15/855,532 filed Dec. 27, 2017, now U.S. Pat. No. 10,292,571;

PCT/US18/14880 filed Jan. 23, 2018;

Ser. No. 16/407,028 filed May 8, 2019, now U.S. Pat. No. 11,253,141;

Ser. No. 16/413,160 filed May 15, 2019, now U.S. Pat. No. 10,869,592;

Ser. No. 16/447,251 filed Jun. 20, 2019, now U.S. Pat. No. 11,013,141;

PCT/US20/38349 filed Jun. 18, 2020;

PCT/US20/46018 filed Aug. 12, 2020;

Ser. No. 17/122,282 filed Dec. 15, 2020;

Ser. No. 17/145,466 filed Jan. 11, 2021, now U.S. Pat. No. 11,395,579;

Ser. No. 17/370,575 filed Jul. 8, 2021;

Ser. No. 17/349,674 filed Jun. 16, 2021; and

Ser. No. 17/573,095 filed Jan. 24, 2022.

### FIELD

This patent specification relates to endoscopy instruments and methods. More particularly, some embodiments relate to portable instruments that include a reusable portion and a releasably attached single-use portion.

### BACKGROUND

Endoscopes have long been used to view and treat internal tissue. In the case of both rigid and flexible conventional endoscopes, the optical system and related components are relatively expensive and are intended to be re-used many times. Therefore, stringent decontamination and disinfection procedures need to be carried out after each use, which adds significant expense in equipment and labor.

In recent years, disposable endoscopes have been developed and improved, typically comprising a single-use portion that includes a cannula with a camera at the distal end. The cannula releasably attaches to a reusable portion that includes image processing electronics and a display. Disposable or single-use endoscopy significantly lessens the risk of cross-contamination and hospital acquired diseases and eliminates the expense and time for decontamination and disinfection that non-disposable endoscopes require. The disposable endoscopes find applications in medical procedures such as imaging and treating the male and female urinary system and the female reproductive system and other internal organs. Examples of disposable or single-use endoscopes are discussed in the patents and application incorporated by reference.

The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### SUMMARY

As described in the initially presented claims but subject to amendments thereof in prosecuting this patent application, according to some embodiments a compact robotic endoscope comprises: a reusable portion that is configured for being grasped by hand and has an elongated slot that has an open side extending along a cannula axis; a single-use portion comprising a cannula that extends along the cannula axis; wherein said single use portion has distal and proximal ends and comprises: an imaging module at the distal end, comprising one or more cameras and one or more light sources; a proximal port at the proximal end, a distal port at the distal end, and an internal channel extending from the proximal port to the distal port; a driven element at the proximal end, configured to selectively change a physical parameter of the cannula when driven; and a single-use electrical contact at the proximal end; wherein said reusable portion comprises: an internal power source; an internal motor; a gear shaft extending into said slot and coupled with the motor to be selectively rotated thereby; a reusable electrical contact that is inside said slot; one or more manually operated motor control buttons coupled with the power source and the motor to cause the motor to drive the gear shaft in selected angular directions; one or more manually operated image control buttons configured to control said imaging module; wherein said proximal end of the single-use portion is configured to fit into said slot of the reusable portion in a direction transverse to the cannula axis to thereby in a single motion mate said electrical contacts with each other and to mate the gear shaft with the driven element and thereby form an assembled endoscope; and wherein in the assembled endoscope: said one or more image control buttons are configured to selectively control transfer of image data from the imaging module for display thereof; and said one or more motor control buttons are configured to selectively control at least one of (a) robotic rotation of the cannula about the cannula axis in a selected angular direction and through a selected angle and (b) robotic angulation of the distal portion of the cannula in a selected direction relative to the cannula axis and through a selected angle relative to the cannula axis.

According to some embodiments, the compact robotic endoscope can additionally comprise one or more of the following: (a) said one or more motor control buttons can be configured to selectively control robotic rotation of the cannula about the cannula axis in a selected angular direction and through a selected angle; (b) said one or more motor control buttons can be configured to selectively control robotic angulation of the distal portion of the cannula in a selected direction relative to the cannula axis and through a selected angle relative to the cannula axis; (c) a display mounted on said reusable portion and coupled with said imaging module and configured to display images acquired therewith; (d) a display mounted on said reusable portion and having a proximal-facing side configured to display said image data and a distally facing side and comprising: a forward-facing camera module (FFC) configured to image an object distally spaced therefrom and comprising autofocus facilities with lenses configured to automatically maintain said object in focus despite relative motion between the FFC; and a forward-facing lighting module (FFL) configured to illuminate said object: (e) said FFC module can comprise at least two cameras spaced apart in a direction transverse to the cannula axis and configured to provide at least one of: (i) stereo images, and (ii) images at wavelength ranges of light that differ as between at least two of said cameras; (f) the reusable portion can further include a latch configured to move between an open position in which the latch clears said slot to enable assembling the single-use and reusable portions by relative movement therebetween in only a direction transverse to the cannula axis and a closed position in which the latch keeps the single-use and reusable portions as an assembled endoscope; (g) an injection needle permanently mounted inside said cannula and configured to move between an extended position in which the injection needle protrudes distally from the distal end of the single-use portion and a retracted position in which the entirety of the injection needle is inside the single-use portion, and a needle control mounted to the single-use portion and configured to move the injection needle between the extended and retracted positions; (h) said cannula can be configured to rotate relative to the distal end of the single-use portion and said driven element at the proximal end of the single-use portion comprises first gear that mounts over the gear shaft in the assembled endoscope to be rotated thereby about an axis transverse to the cannula axis, a second gear engaging the first gear to be rotated thereby about an axis parallel to the cannula axis, a third gear affixed to the second gear to rotate therewith, and a fourth gear mating with the third gear to be rotated thereby about the cannula axis and thereby rotate the distal end of the cannula about the cannula axis relative to the proximal end of the single-use portion; and (i) said distal end of the cannula can be configured to angulate through said selected angle and said driven element at the proximal end of the single-use portion comprises a rotary disc that mounts over the gear shaft in the assembled endoscope to be rotated thereby about an axis transverse to the cannula axis and said single use portion comprises guide wires coupling the rotary disc to the distal end of the cannula such that rotation of the rotary disc causes the distal end of the cannula to angulate relative to the cannula axis.

According to some embodiments, a compact robotic endoscope comprises a set of a reusable portion and two different single-use portions a first of which has a motor-driven cannula rotation and a second has a motor-driven angulation of a distal end of a cannula, wherein: the reusable portion is configured for being hand-held and has an elongated slot that has an open side extending along a cannula axis, has an internal motor and a gear shaft extending into said slot and configured to be selectively rotated by the motor about a shaft axis, and an electrical contact in said slot; the first single-use portion is configured to be coupled with said reusable portion by being snapped into said slot solely in a direction transverse to the cannula axis to thereby form a first assembled endoscope, and comprises a distal end with an imaging module, a proximal end with a socket configured to fit over said gear shaft to be rotated thereby about the shaft axis in the first assembled endoscope and a gear set coupling the socket with the distal end of the first single-use portion to impart rotation thereof relative to the proximal end thereof about the cannula axis in a selected angular direction and to a selected degree; the second single-use portion also is configured to be coupled with said reusable portion by being snapped into said slot solely in a direction transverse to the cannula axis to thereby form a second assembled endoscope, and comprises a distal end with an imaging module, a proximal end with a socket configured to fit over said gear shaft to be rotated thereby about the shaft axis in the second assembled endoscope and guide wires coupling the socket with the distal end of the second single-use portion to angulate said distal of the distal end of the second single-use portion relative to the proximal end thereof in a selected direction and to a selected degree relative to the cannula axis; thereby enabling the same reusable portion to form the first endoscope in which the distal end of the first single-use portion robotically rotates or the second endoscope in which the distal end of the single-use portion robotically angulates.

According to some embodiments, a set comprises a reusable portion and two different single-use portions, in which the proximal end of the first single-use portion further comprises a manual control coupled with said distal end of the first single-use portion to impart angulation thereof relative to the proximal end of the first single-use portion in a selected direction and to a selected degree relative to the cannula axis; (b) the proximal end of the second single-use portion can further comprise a manual control coupled with said distal end of the second single-use portion to impart rotation thereof relative to the proximal end of the second single-use portion in a selected direction and to a selected degree about the cannula axis; and (c) the reusable portion can furthest comprise a display mounted thereof and having a proximal face configured to display images acquired with said imaging module and a distal face with a light source and one or more forward-facing cameras (FFC) configured to image an object distal therefrom and provided with autofocus on said object as the endoscope moves relative to the object.

According to some embodiments, a robotic endoscope that is single-use in its entirety comprises: a handle that has a distal end and a proximal end and is configured to be hand-grasped by a user and further has a working channel port at the proximal end thereof, an internal motor with a motor shaft that rotates when the motor is energized, and buttons configured to selectively energize the motor to rotate the motor shaft in a selected angular direction and through a selected angle; a hub that has a proximal end permanently mounted to the distal end of the handle to rotate relative thereto about a cannula axis; a cannula that has a proximal end permanently secured to the distal end of the hub, extends therefrom along the cannula axis, and has an imaging module and a working channel port at a distal end thereof; a working channel with constant internal area, extending from the working channel port at the proximal end of the handle to the working channel port at the distal end of the cannula; coupling elements connecting said motor shaft to the distal end of the cannula and configured to translate rotation of the motor shaft in a selected angular direction through a selected angle to angulation of the distal end of the cannula in a selected direction and to a selected degree relative to the cannula axis; and a transmission facility coupled to said imaging module to transmit images acquired thereto to a display outside the single-use endoscope.

According to some embodiments, the robotic endoscope described in the immediately preceding paragraph can further include one or more of the following: (a) a remote display and control facility coupled with said transmission facility and configured to control said imaging module and to display images acquired with the imaging module; and (b) said transmission facility can be wireless.

According to some embodiments, an endoscope that is single use in its entirety comprises: a handle that has a distal end and a proximal end and is configured to be hand-grasped by a user and further has a working channel port at the proximal end thereof, and a lever configured to be manually moved between first and second positions relative to the handle; a cannula that has a proximal end permanently secured to the distal end of the hub, extends therefrom along the cannula axis, and has an imaging module and a working channel port at a distal end thereof; a working channel with constant internal area, extending from the working channel port at the proximal end of the handle to the working channel port at the distal end of the cannula; a motion translating mechanism inside the handle that is coupled to the lever and to the distal end of the cannula and is configured to translate motion of the lever in a selected direction and to a selected degree to angulation of the distal end of the cannula in a selected direction and to a selected degree relative to the cannula axis; and a transmission facility coupled to said imaging module to transmit images acquired thereto to a display outside the single-use endoscope.

According to some embodiments, the robotic endoscope described in the immediately preceding paragraph can further include one or more of the following: (a) the lever can be configured to pivot about an axis transverse to the cannula axis and the motion translation mechanism comprises an element coupled with the lever to rotate with pivoting of the lever and guide wires connecting the element to the distal end of the cannula; and (b) a remote display and control facility coupled with said transmission facility and configured to control said imaging module and to display images acquired with the imaging module.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail using the accompanying drawings in which:

FIG. 1A is a perspective view of a first example of an assembled endoscope, with a cannula that robotically rotates, FIG. 1B is a perspective exploded view of a right side of the unassembled endoscope example showing a reusable portion and a single-use portion thereof, FIG. 1C is a partial exploded perspective view of a left side of a the single-use portion thereof, and FIG. 1D is a perspective exploded view of a left side of the unassembled endoscope example, according to some embodiments;

FIG. 2A is a perspective view of a right side of a second example of an assembled endoscope, with a cannula that robotically angulates, FIG. 2B is a perspective exploded view of a right side of the second endoscope example when unassembled, FIG. 2C is an exploded perspective view of a left side of the second endoscope example when unassembled, FIG. 2D is an elevation view of a left side of a proximal part of the single-use portion of the second endoscope example, and FIG. 2E is a perspective exploded view of a mechanism involved in angulating the distal end of a cannula of the second endoscope example, according to some embodiments;

FIG. 3 is a partial perspective view of the right side of a third endoscope example that can be otherwise like the endoscope example of FIG. 1A or the endoscope example of FIG. 2A but adds an illumination source at a distal face of a display providing light beams having respective selected wavelength ranges and further adds forward-facing cameras (FFC) that can include autofocus facilities, according to some embodiments.

FIG. 4A is perspective view of a right side of a fourth endoscope example that is single use in its entirety, has a cannula that both angulates and rotates under manual control, and is designed for use with a remote display, FIG. 4B is a perspective view of a left side thereof, and FIG. 4C is an exploded perspective view of components thereof, according to some embodiments.

FIG. 5A is perspective view of a right side of a fifth endoscope example that is single use in its entirety, has a cannula that angulates by robotic control and rotates under manual controls, and is designed for use with a remote display, FIG. 5B is a perspective view of a left side thereof, and FIG. 5C is an exploded perspective view of components thereof, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein. It should be clear that individual features of one or several of the specific embodiments described herein can be used in combination with features of other described embodiments or with other features.

Like reference numbers and designations in the various drawings indicate like elements. Further, the reference numbers of components that are like in structure and function have the same second and third digits. For conciseness, components that bear the same reference numbers of the same last two digits of reference numbers are described only in connection with the Figure that first refers to them and the description is not repeated in connection with subsequently discussed Figures.

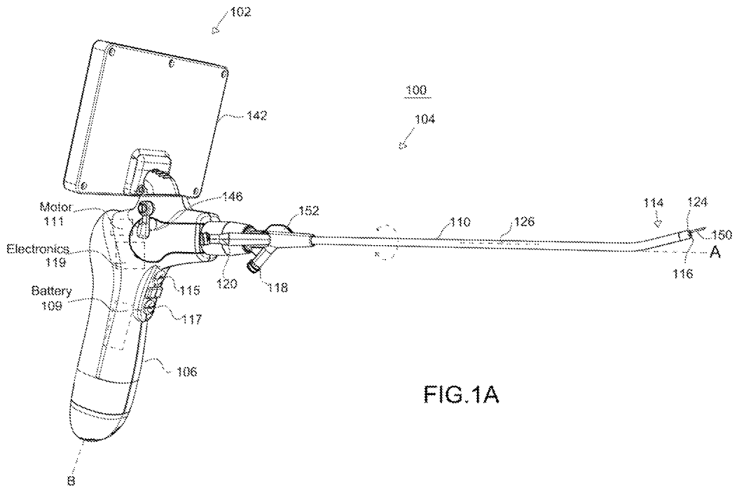

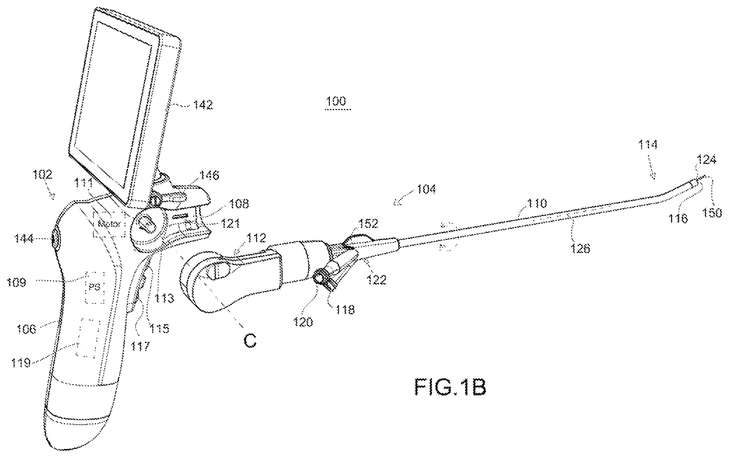

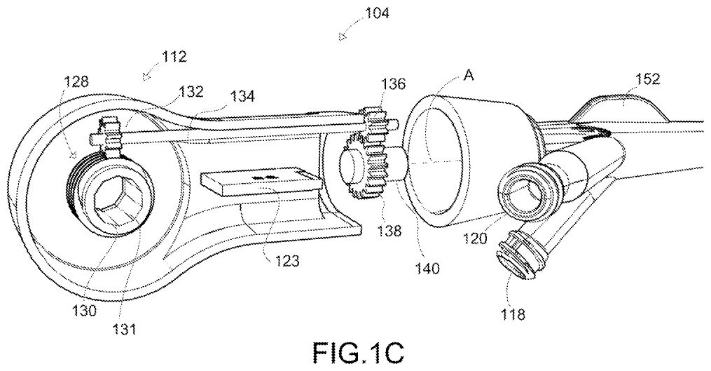

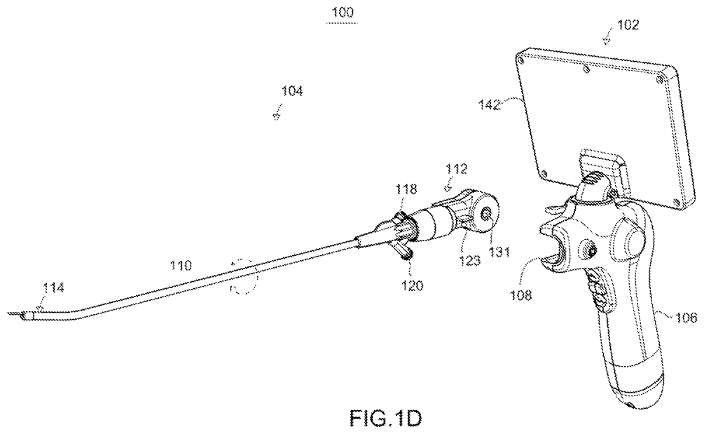

FIGS. 1A-1D illustrate a first example of an endoscope. This example robotically controls motorized rotation of a cannula about its long axis relative to a proximal portion of a single-use portion and a reusable portion to which the single-use portion releasably attaches to form an assembled endoscope, according to some embodiments.

FIG. 1A is a perspective view of a right side of an assembled endoscope 100, FIG. 1B is a perspective exploded view of a right side of endoscope 100 when unassembled, showing a reusable portion 102 and a single-use portion 104 thereof, FIG. 1C is an exploded partial perspective view of a left side of single-use portion 104 thereof, and FIG. 1D is a perspective exploded view of a left side of endoscope 100 when unassembled, according to some embodiments.

As seen in FIGS. 1A-1D, reusable portion 102 comprises a handle 106 that extends along a handle axis B and is configured to be hand-held. Reusable portion 102 has an elongated slot 108 seen in FIG. 1B that has an open side facing to the right and extends along a cannula axis A that is transverse to handle axis B. Single-use portion 104 comprises a cannula 110 that extends along the cannula axis A and has a proximal end 112 that releasably attaches to reusable portion 102 at slot 108 and a distal end 114 tipped with an imaging module 116 that comprises one or more cameras and one or more light sources. One or more proximal ports 118, 120 are at proximal end 122 of cannula 110, one or more distal ports 124 are at distal end 114 of cannula 110, and one or more internal channels 126 (schematically shown by a dashed line) extend from proximal ports 118, 120 to distal port(s) 124 to enable fluid flow between the proximal and distal ports through channel(s) 126 and/or to enable surgical tools to be inserted into one or more of proximal ports 118, 120, pass through channel(s) 126, and protrude distally from one or more of distal ports 124.

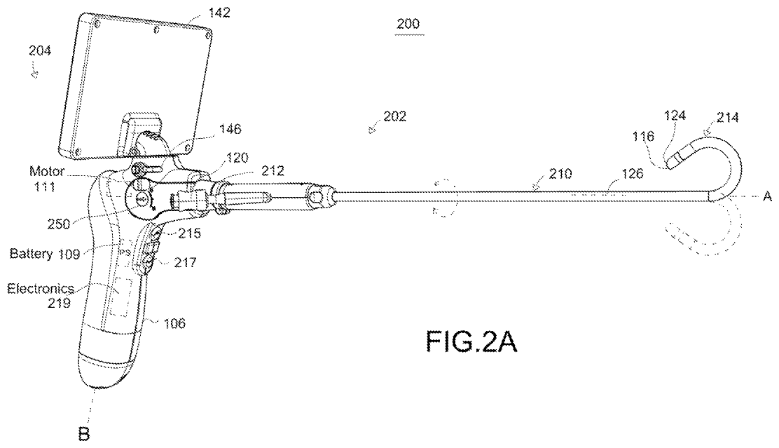

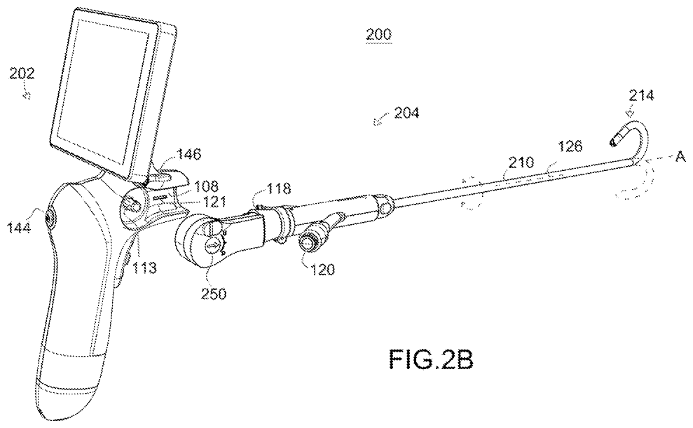

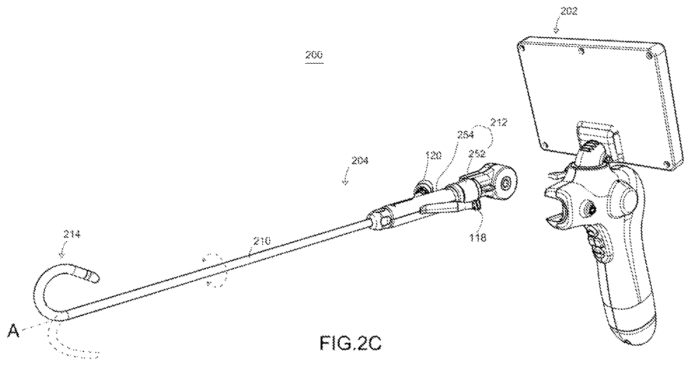

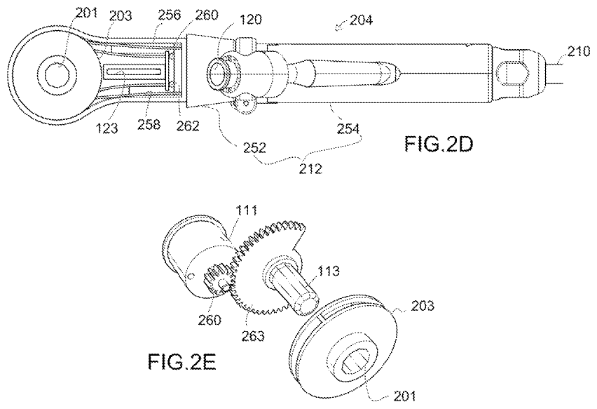

As seen in FIG. 1B, reusable portion 102 comprises an internal power source 109 such as a battery and an internal motor 111 that are schematically illustrated. A gear shaft 113 extends into slot 108, along an axis C that is perpendicular to cannula axis A and handle axis B and is coupled with motor 111 to be selectively rotated thereby about axis C clockwise and counterclockwise as needed over selected angles of rotation. Motor 111 can be a step motor and can be directly behind gear shaft 113 such that the motor shaft rotates about an axis parallel to axis C. Motor 111 and gear shaft 113 are shown in perspective view in FIG. 2E and explained in more detail further below. Gear shaft 113 has a polygonal outer surface configured to engage and drive a cylinder 130 (FIG. 1C) in single-use portion 104, as described further below, when endoscope 100 is assembled. Handle 106 has at its distal face push buttons 115 and 117 that selectively couple power source 109 with motor 111 to robotically cause gear shaft 113 to rotate in a selected angular direction over a selected angle. Buttons 115, 117 preferably are positioned to be operated with the forefinger and/or middle finger of a user grasping handle 106.

Reusable portion 102 preferably includes a display 142 mounted thereon and coupled with imaging module 116. Both preferably are powered with power source 109. In addition, reusable portion 102 has a button 144 at the proximal end thereof positioned for operation with the thumb of a user grasping handle 106. Button 144 is operatively coupled with imaging module 116 at the distal end 114 of cannula 110 to control operations of the imaging module such as imaging functions and may be coupled with display 142 to control display functions. Button 144 can comprise two or more buttons or another suitable interface to control respective functions of imaging module 116 and/or of display 142. Reusable portion 102 further includes a pivoting latch 146 shown in a closed position in FIG. 1A to hold reusable portion 102 and single-use portion 104 together, and in an open position shown in FIG. 1B to allow snapping single-use portion 104 into slot 108 of reusable portion 102.

Reusable portion 102 preferably further include electronics 119 schematically shown in FIG. 1A and coupled with imaging module 116 and/or display 142 and if desired with buttons 115, 117, and 144 to facilitate one or more of: processing image data from and controlling imaging module 116, controlling motor 111, and controlling display 142. An electrical contact 121 (FIG. 1B) in slot 108 of reusable portion 102 is configured to mate with an electrical contact 123 FIG. 1C) of single use portion 104 as described below.

Single-use portion 104 comprises a driven element 128 inside proximal end 112 of single-use portion 104 that mates with gear shaft 113 when the reusable portion 102 and single-use portion 104 are assembled into the configuration seen in FIG. 1A. Details of driven element 128 are seen in FIG. 1C. As shown, driven element 128 comprises a cylinder 130 that has a polygonal internal surface forming a socket and is helically threaded externally. Cylinder 130 fits over gear shaft 113 (FIG. 1B) when endoscope 100 is assembled as seen in FIG. 1A. The polygonal internal surface 131 of cylinder 130 matches the extremal polygonal surface of gear shaft 113 (FIG. 1B) to be rotated by gear shaft 113 in the assembled endoscope. Driven mechanism 128 is operatively coupled with canula 110 to selectively rotate the cannula about cannula axis A relative to proximal end 112 of single-use portion 104 to selectively change a physical parameter of the cannula when driven, namely, the angular position of the cannula relative to proximal end 112 and reusable portion 112. Driven element 128 comprises, in addition to cylinder 130, a gear 132 that meshes with the external helical thread of cylinder 130 to be rotated thereby about an axis parallel to cannula axis A, a shaft 134 affixed at its proximal end to gear 132, another gear 136 that is affixed to the distal end of shaft 134, a gear 138 that meshes with gear 136 to be rotated thereby about cannula axis A and a shaft 140 that is affixed to gear 138, extends distally therefrom, and if affixed to a proximal end of cannula 110 to selectively rotate the cannula about cannula axis A relative to proximal end 112 of single-use portion 104. Single-use portion 104 further includes an electrical contact 123 (FIG. 1C) configured to mate with electrical contact 121 (FIG. 1B) in reusable portion 102 to thereby transmit power, controls, and image data between the reusable and single-use portions when endoscope 100 is in its assembled configuration seen in FIG. 1A.

FIG. 1D is a view of the left side of unassembled endoscope100 and shows that the left side of proximal end 112 of single-use portion 102 is enclosed in a housing that has an opening into the internal surface 131 of cylinder 130.

According to some embodiments, endoscope 100 permanently encloses an injection needle 150 that moves between an extended position in which a distal end of the needle protrudes distally from distal end 114 of cannula 110 and a retracted position in which the distal end of needle 150 is substantially or entirely inside cannula 110. Injection needle 150 can be moved manually between its positions with a knob 152 that is affixed to a proximal end of needle 150 and is configured to slide relative to proximal end 112 of single-use portion 104. Fluid can be injected into needle 150 through one of ports 118, 120 when the port is configured in fluid-flow communication with needle 150. For details on how to incorporate an injection needle in a single-use portion of an endoscope, see for example U.S. Pat. Nos. 10,524,636 and 10,874,287 incorporated by reference.

In operation, single-use portion 104 is supplied to a user sealed in a sterile package. The user tears up the package and assembles single-use portion 104 with reusable portion 102 into the configuration of endoscope 100 seen in FIG. 1A by snapping proximal portion 104 into slot 108 such that gear shaft 113 fits into the polygonal opening (socket) of cylinder 130 and electrical contacts 121 and 123 mate to establish electrical connection. No tools are required for assembling endoscope 100. The user then latches portions 102 and 104 to each other by pivoting latch 146 from the open position seen in FIG. 1B to the closed position seen in FIG. 1A. The user can grasp and hold handle 106 and insert cannula 110 into a patient, for example toward or into urinary or reproductive organs. Cannula 110 can be robotically rotated relative to handle 106, with the angular direction and extent of rotation controlled by manual operation of buttons 115, 117. Because the distal end 114 is angled from axis A as seen in FIGS. 1A and 1B, rotation of the cannula enables viewing an internal organ from different directions. For example, all or nearly all the internal wall of a bladder can be examined with imaging module 116 by such rotation of cannula 110. According to some embodiments, the degree of rotation of cannula 110 can be limited, for example to half-circle or less in each direction, by suitable mechanical stops that prevent rotating cylinder 130 by more than a selected angle in each direction (and the angles can be different in different angular directions), or by electronically controlling motor 111 through electronics 119. After endoscope 100 is used in a medical procedure, the user moves latch 146 to its open position, pulls single-use portion 102 away from reusable portion 104 by hand, without tools, and discards the used single-use portion per procedures for handling medical waste. For other examples of rotating a cannula see U.S. Pat. Nos. 9,895,048, 11,350,816 and 11,330,973 and U.S. patent application Ser. Nos. 17/745,526 and 17/835,624 incorporated by reference.

FIGS. 2A-2E illustrate an endoscope 200 that robotically controls motorized angulation of a distal end of a cannula that is a part of a single-use endoscope portion and a reusable portion to which the single-use portion releasably attaches to form an assembled endoscope, according to some embodiments.

FIG. 2A is a perspective view of a right side of a second assembled endoscope 200, FIG. 2B is a perspective exploded view of a right side of the endoscope 200 when unassembled, FIG. 2C is an exploded perspective view of a left side of endoscope 200 when unassembled, FIG. 2D is a view of a left side of a proximal end of the single-use portion of endoscope 200, and FIG. 2E is a perspective exploded view of a mechanism involved in angulating the distal end of a cannula of endoscope 200, according to some embodiments.

FIG. 2A shows assembled endoscope 200 in which reusable portion 202 can be like reusable portion 202 in endoscope 100 except that: (a) buttons 215, 217 control angulation of a distal end 214 of a cannula 214, (b) electronics 219 control parameters related to angulation of distal end 214 and in this respect replace electronics 119 regarding control of cannula rotation, and (c) mechanical of electronic stops can limit the extend of angulation. Single-use portion 204 is otherwise like portion 102 of endoscope 100 except that: (a) gear shaft 113 engages and rotates a disc 203 with a polygonal central opening (socket) 201 (FIG. 2C), which is in place of driven element 128 in endoscope 100, (b) guide wires or cables convey angulation forces to the distal end 214, (c) a control 250 can be provided for directions of angulation, (d) rotation of cannula 210 relative to proximal end 212 of single-use portion 204 is controlled manually, and (e) no internal injection needle is included in the illustrated example.

Referring to FIG. 2A, buttons 215, 217 control the direction and extent of angulation of distal end 214 of cannula 210. For example, button 215 when pressed causes angulation that curls up distal end 214, to the degree shown in FIG. 1A or to a lesser of greater degree depending on how long button 215 remains depressed with the user's finger, and button 217 causes angulation of distal end 214 in the opposite angular direction such that distal end 214 curls down, to the degree shown in dash lines or to a lesser or greater degree depending on how long button 217 remains pressed. FIG. 2A shows latch 146 in an open position but in practice would be in the closed position when endoscope 200 is assembled and ready for use in a medical procedure.

FIG. 2B shows the right sides of reusable portion 202 and single-use portion 204, which can be assembled into endoscope 200 as discussed above for portions 104 and102. Single-use portion 204 can include a 3-position manually operated switch 250 that a user can rotate to a position U to limit the direction of angulation to curling up from cannula axis A, a position D to limit the direction of angulation to curling down from cannula axis A, and an intermediate portion in which distal end 214 can curl up or down depending on which button 215, 217 the user depresses.

FIG. 2C shows the left sides portions 204 and 202 of unassembled endoscope 200. In external appearance, in this view they are like portions 104 and 102 seen in FIG. 1D except for the difference between distal ends 114 and 214. As illustrated in FIG. 2C, proximal end 212 of single-use portion 204 comprises a proximal part 252 that does not rotate relative to reusable portion 202 in the assembled endoscope 200 and a part 254 that is rotatably mounted to part 252 to rotate relative to part 252 about cannula axis A. Cannula 210 is affixed to part 254 to rotate therewith. In the example of endoscope 200, rotation of cannula 210 is manual—for example the user grasps portion 254 or ports 118, 120 and rotates cannula 210 in the desired angular direction and to the desired degree. The maximum degree of rotation can be limited by mechanical stops in non-rotatable part 252, for example to half-circle or less in each angular direction.

FIG. 2D is a plan, partly cut-open view from the left of a part of single-use portion 202 of endoscope 200. Guide wires or cables 256 and 258 are anchored at fixed locations 260 and 262 in single-use portion 204, looped over disc 203, and anchored at locations (not shown) at distal end 214 of cannula 210 such that rotation of disc 203 in one angular direction pulls one of cables 256 and 258 and slackens the other and thus angulates distal end 214 in one angular direction. Rotating disc 203 in the opposite angular direction has the opposite effect on distal end 214. As noted, mechanical stops can be mounted to engage disc 203 as needed to prevent rotation in an angular direction by more than selected angles. The mechanical stops can limit rotation of disc 203 in each direction to the same angle or can limit rotation of disc 203 to different angles in the different angular directions.

FIG. 2E is an exploded perspective view of elements of reusable portion 202 that can be like in endoscopes 100 and 200 and elements of disc 203 of single-use portion 204 that differ from single-use portion 102. Motor 111 is fixedly mounted in reusable portion 102 and 202, to the left of gear shaft 113 and drives gear shaft 113 through meshing gears 260, 262 in an angular direction and to a degree selected by buttons 115, 117 and electronics 119 in endoscope 100 or buttons 215, 217 and electronics 219 in endoscope 200. As shown in FIG. 2E, gear shaft 113 fits in opening (socket) 201 in disc 203 such that in assembled endoscope 200 disc 203 rotates with gear shaft 113. The polygonal inside shape of opening (socket) 201 closely matches the polygonal circumference of gear shaft 113 to ensure common rotation, ease of mounting disc 203 on shaft 113 and of dismounting the disc, and sufficiently close fit to prevent undesirable slippage or vibration at start, stop and reversal of rotation. Cylinder 130 of single-use portion 102 similarly fits over gear shaft 113. For another example of an endoscope in which the cannula rotates and has a distal end that angulates see U.S. Pat. No. 10,292,571 incorporated by reference.

In each endoscope 100 and 200, motor 111 can be a step motor. In endoscope 100, motor 111 can be coupled with electronics 119 to be controlled thereby and to enable electronics 119 to keep a count of the steps of motor 111, which steps are a measure of the angle by which motor 11 has rotated cannula 110 about axis A in the respective angular direction that also is controlled by electronics 119. Thus, the count kept in electronics 119 can be a metric that electronics 119 can use to control parameters such as how images from imaging module 116 are displayed on display 142. In endoscope 200, electronics 219 can similarly count the motor steps as a metric of how far in in what direction distal end 214 has angulated.

In one example of endoscope 100, when cannula 110 rotates and therefore imaging module 116 provides images angled differently to fixed vertical and horizontal planes, e.g., angled differently relative to the room in which endoscope 100 is used in a medical procedure, the images on display 142 rotate the same way relative to display 142. This mode of displaying images can be called a “rotating image” mode. However, some professionals such as gynecologists may have become accustomed to images that they see with an older style rigid endoscope that works like a conventional telescope in that the view does not rotate with rotation of the rigid endoscope about its long axis. In endoscope 100, like rotation of the image on display 142 can be provided by using the metric of counts of steps of motor 111 to rotate the image on display 142. To this end, electronics 119 are configured to cause the image on display 142 to rotate in the same direction and to the same degree relative to display 142 as cannula 110 rotates relative to proximal end 112 of single use portion 102, using the count of motor steps and known image processing technology developed for rotating images on a computer screen. Endoscope 100 thus can be provided with a “keep upright” mode to keep the image on display 142 in the same angular orientation relative to display 142 as the angular orientation of the organ that the endoscope is currently viewing relative to the room. In some embodiments, the “keep upright” mode can be the only way to display images with endoscope 100. In other embodiments, endoscope 100 can be provided with both the “rotating image” mode and the “keep upright” mode and the endoscope can be provided with a switch to enable the user to select one of the modes for a procedure on a patient.

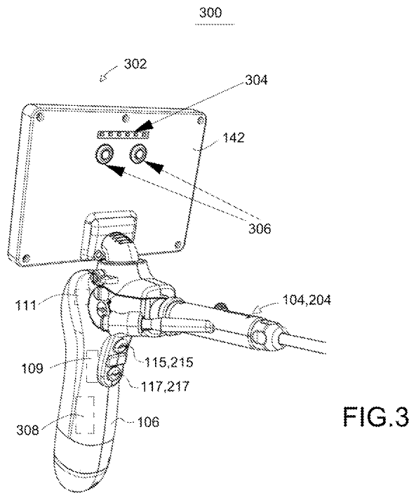

FIG. 3 is a partial perspective view of the right side of an endoscope 300 that can be otherwise like endoscope 100 or endoscope 200 but has at a distal face of display 302, a light source 304 providing light beams having respective selected wavelength ranges and further having forward-facing cameras (FFC) 306 that can include autofocus facilities and has electronics 308 configured to operate with said illumination source and FFCs, according to some embodiments. Light source 304 can comprise a bank of LEDs emitting light at respective selected wavelength ranges, for example white light, blue light, green light, etc. FFCs 306 can comprise two or more cameras configured to image light in the same or respective different wavelength ranges. For other examples of FFCs in reusable portions of endoscopes see U.S. patent application Ser. Nos. 17/473,587 and 17/835,624 incorporated by reference.

FFCs 306 can be provided with autofocus facilities that can be the type used in contemporary smartphones, and with lenses that can be electronically focused for varying object heights and working distances. The lenses of FFCs 306 can be liquid lenses, which are small, mechanically of electrically controlled cells containing optical-grade liquid. When a current or voltage is applied to a liquid lens cell, the shape of the cell changes. This change occurs within milliseconds and causes the optical power, and therefore focal length and working distance to the object being imaged, to shift. In the embodiment of FIG. 3, the lenses of FFCs 306 can be controlled by the autofocus facilities of the FFCs. Alternatively, the FFCs 306 can be provided with lenses configured to move relative to the camera's image plane using Voice Coil Motor working principle that involves a permanent magnetic field and controlling a spring by changing DC current of the coil, thereby driving the lens toward or away from the image plane. Voice Coil Motor (VCM) is widely used in contemporary camera modules. Another option is the poLight® TLens technology that is believed to out-compete many traditional VCM features. A migration from VCM technology to the new poLight® technology is foreseen for smartphones, wearables, consumer devices as well as for demanding industrial or medical applications. The fundamental structure of the TLens® is very different from the VCM technology: Whereas the VCM technology moves the lens barrel to create focus, the TLens® does not require the lens barrel to move but only gets attached to it opto-mechanically, then creates the focusing lens effect to tune the optical characteristics of the combined lens stack.

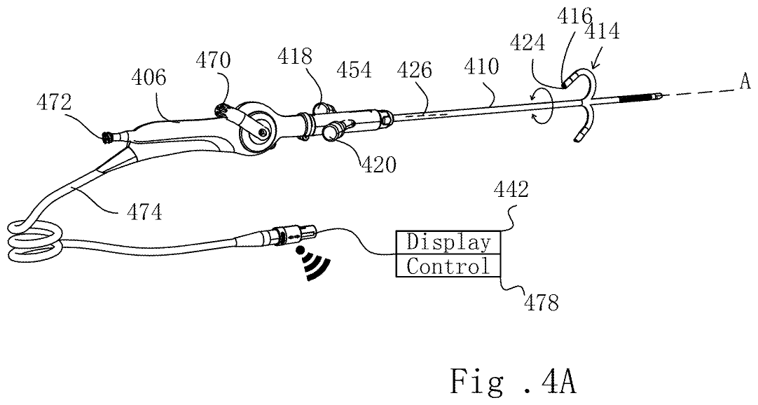

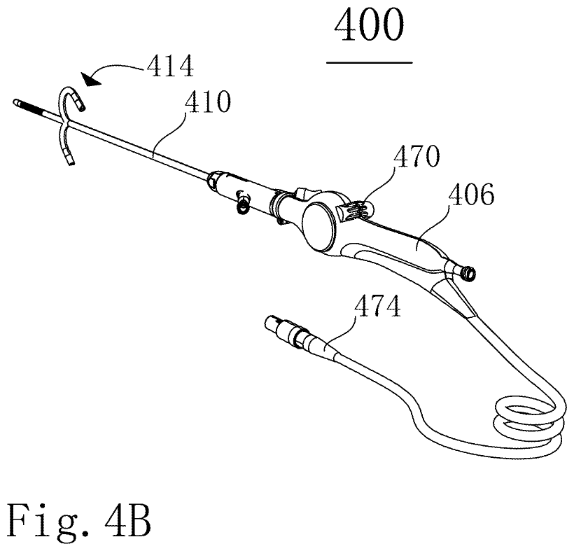

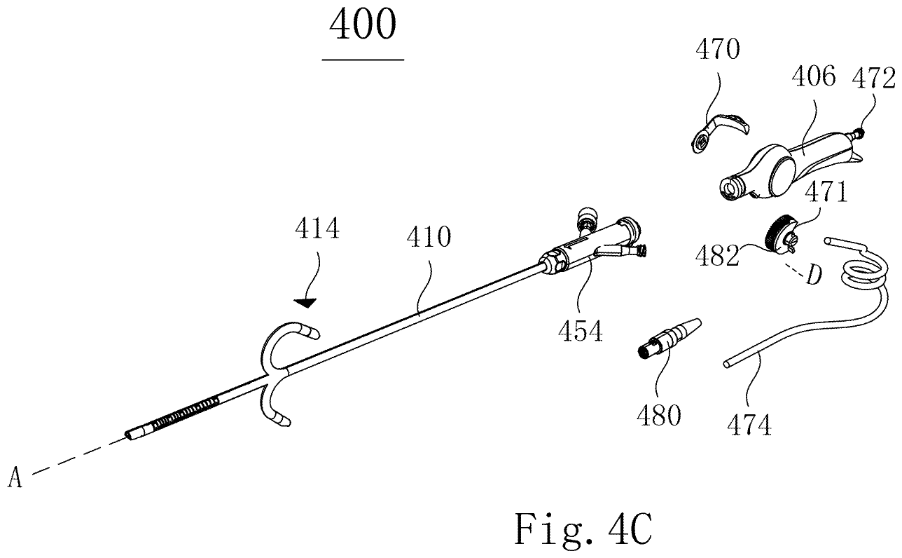

FIG. 4A is perspective view of a right side of an endoscope 400 that is single use in its entirety, has a cannula 410 that both angulates and rotates under manual control, and is designed for use with a remote display 442, FIG. 4B is a perspective view of a left side thereof, and FIG. 4C is an exploded perspective view of components thereof, according to some embodiments.

Endoscope 400 comprises a hub 454 with ports 418, 420 that connect with one or more internal channels 426 in cannula 410 that extend to one or more ports 424 at distal end 414 of the cannula. An imaging module 416 is located at distal end 414. Distal end 414 is configured to angulate in a selected angular direction and to a selected degree under control of lever 470 that pivots around axis D of a pin 471 (FIG. 4C). A working channel port 472 at the proximal end of handle 406 connects to a working channel that extends distally from port 472, through straight handle 406, through hub 454, through cannula 410 (and can be one of channels 426) and ends at one of ports 424. Preferably, the working channel is straight and with the same diameter from port 472 to the cannula curve at distal end 414 and is straight and with the same diameter all the way to distal ports 424 when distal end 414 is not angulated. A cable 474 can connect endoscope 400 to an external display 442, or the connection can be wireless such as via WiFi as indicated by a WiFi symbol. Control 478 can be integrated with display 442 or can be a separate device that connects with imaging module 416 via cable 474 and wires in endoscope 400 and is coupled with display 442 to control imaging and display functions. Endoscope 400 can be manufactured relatively inexpensively as it need not include a power source or image processing electronics. Power can be supplied from an external source, such as display 442 and/or control 478 or a separate source, and image processing can be done by control 478 and/or display 442.

Cannula 410 is affixed to hub 454, which is rotatably mounted to handle 406 to rotate about cannula axis A relative to handle 406. As seen in FIGS. 4A and 4B, a lever 470 can be pivoted about pin 471 manually, typically with the thumb of a user grasping handle 406.

FIG. 4C is an exploded perspective view of components of endoscope 400 and shows a unit comprising cannula 410 and hub 454 that rotatably mounts to the distal end of handle 406 though a connecting element 480 to rotate together with cannula 410 about axis A relative to handle 46. Handle 406 encloses a half wheel 482 that is mounted for rotation with pin 471 about axis D, and lever 470 mounts to pin 471 to rotate therewith about axis D. Guide wires or cables (not shown) connect half wheel 482 to distal end 414 to angulate distal end 414 much like described for endoscope 200. The function of half wheel 482 like that of disc 203.

The entire endoscope 400 is supplied to users in a sealed sterile packaging. For a medical procedure, the user tears the packaging open and connects cable 474 to remote display 442 and/or control 478 or establishes a wireless connection. The user controls imaging module 416 through display 442 and/or control 478, which are configured to carry out the required image processing that can be as described above for endoscopes 100, 200, and 300. After the medical procedure the entire endoscope 400 is disposed of as medical waste.

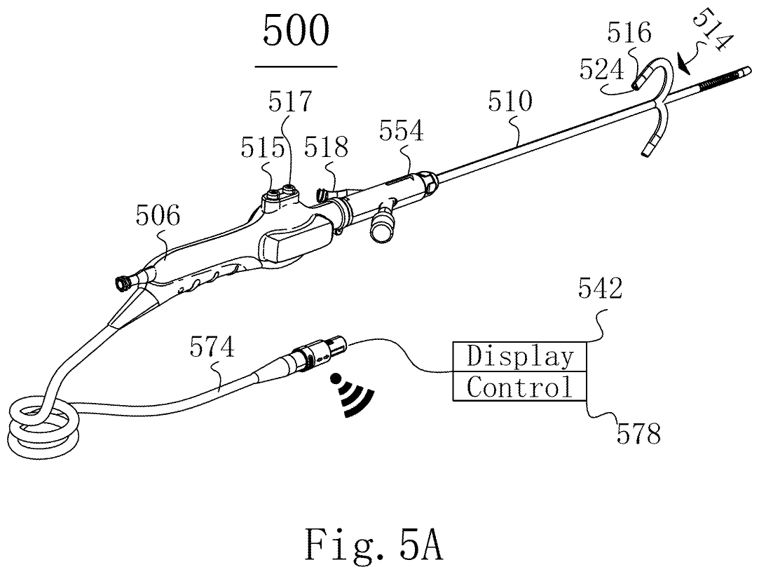

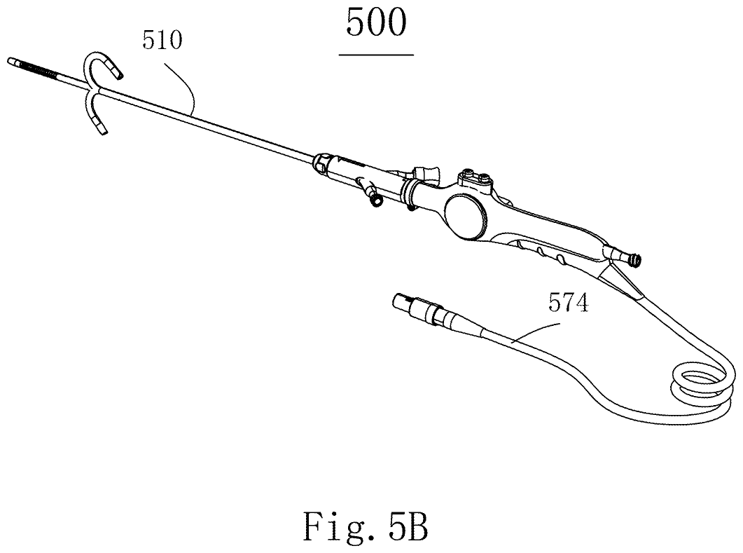

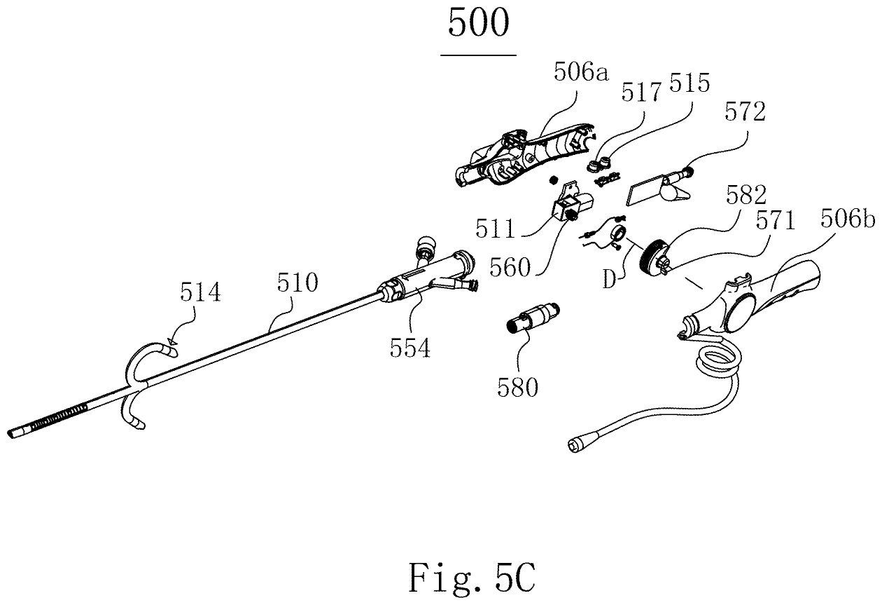

FIG. 5A is a perspective view of a right side of an endoscope 500 that is otherwise like endoscope 400 but angulation of distal end 514 of cannula 510 is robotically controlled by a motor 511 (FIG. 5C) and buttons 515, 517 and the mechanism of transmitting motion from the motor to guide wires and cables is different. FIG. 5B is a perspective view of a left side thereof, and FIG. 5C is an exploded perspective view of components thereof, according to some embodiments.

The components that are distal from handle 506 can be like those distal from handle 406. Corresponding components differ only by the first digit of their reference numerals. Handle 506 differs from handle 406 only in that handle 506 encloses a motor 511 (FIG. 5C) that rotates a plate 584 under control of buttons 515, 517, in place of the manual control over angulation of endoscope 400.

Referring to FIG. 5C, handle 506 is formed by half-shells 506a and 506b and encloses a motor 511 that rotates a gear shaft 560 about axis D. Half wheel 582 fits over gear shaft 560 to rotate therewith and plate 584 is secured to half wheel 582 to rotate therewith about axis D. Guide wires or cables (not shown) are secured to plate 584 such that rotation of plate 584 controls angulation of distal end 514 of cannula 510 in the manner described above for endoscopes 200 and 400.

Although the foregoing has been described in some detail for purposes of clarity, it will be apparent that certain changes and modifications may be made without departing from the principles thereof. There can be many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. A compact robotic endoscope comprising:
a reusable portion that is configured for being hand-held and has an elongated slot that has an open side extending along a cannula axis;
a single-use portion comprising a cannula that extends along the cannula axis;
wherein said single use portion has distal and proximal ends and comprises:
an imaging module at the distal end, comprising one or more cameras and one or more light sources;
a proximal port at the proximal end, a distal port at the distal end, and an internal channel extending from the proximal port to the distal port;
a driven element at the proximal end, configured to selectively change a physical parameter of the cannula when driven; and
a single-use electrical contact at the proximal end;
wherein said reusable portion comprises:
an internal power source;
an internal motor;
a gear shaft extending into said slot and coupled with the motor to be selectively rotated thereby;
a reusable electrical contact that is inside said slot;
one or more manually operated motor control buttons coupled with the power source and the motor to cause the motor to drive the gear shaft in selected angular directions;
one or more manually operated image control buttons configured to control said imaging module;
wherein said proximal end of the single-use portion is configured to fit into said slot of the reusable portion in a direction transverse to the cannula axis to thereby in a single motion mate said electrical contacts with each other and to mate the gear shaft with the driven element and thereby form an assembled endoscope; and
wherein in the assembled endoscope:
said one or more image control buttons are configured to selectively control transfer of image data from the imaging module for display thereof; and
said one or more motor control buttons are configured to selectively control at least one of (a) robotic rotation of the cannula about the cannula axis in a selected angular direction and through a selected angle and (b) robotic angulation of the distal portion of the cannula in a selected direction relative to the cannula axis and through a selected angle relative to the cannula axis.

2. The compact robotic endoscope of claim 1, in which said one or more motor control buttons are configured to selectively control robotic rotation of the cannula about the cannula axis in a selected angular direction and through a selected angle.

3. The compact robotic endoscope of claim 1, in which said one or more motor control buttons are configured to selectively control robotic angulation of the distal portion of the cannula in a selected direction relative to the cannula axis and through a selected angle relative to the cannula axis.

4. The compact robotic endoscope of claim 1, further including a display mounted on said reusable portion and coupled with said imaging module and configured to display images acquired therewith.

5. The compact robotic endoscope of claim 1, further including a display mounted on said reusable portion and having a proximal-facing side configured to display said image data and a distally facing side and comprising:
a forward-facing camera module (FFC) configured to image an object distally spaced therefrom and comprising autofocus facilities with lenses configured to automatically maintain said object in focus despite relative motion between the FFC and
a forward-facing lighting module (FFL)configured to illuminate said object.

6. The compact robotic endoscope of claim 5, in which said FFC module comprises at least two cameras spaced apart in a direction transverse to the cannula axis and configured to provide at least one of: (a) stereo images, and (b) images at wavelength ranges of light that differ as between at least two of said cameras.

7. The compact robotic endoscope of claim 1, in which the reusable portion further includes a latch configured to move between an open position in which the latch clears said slot to enable assembling the single-use and reusable portions by relative movement therebetween in only a direction transverse to the cannula axis and a closed position in which the latch keeps the single-use and reusable portions as an assembled endoscope.

8. The compact robotic endoscope of claim 1, further including an injection needle permanently mounted inside said cannula and configured to move between an extended position in which the injection needle protrudes distally from the distal end of the single-use portion and a retracted position in which the entirety of the injection needle is inside the single-use portion, and a needle control mounted to the single-use portion and configured to move the injection needle between the extended and retracted positions.

9. The compact robotic endoscope of claim 1, in which said cannula is configured to rotate relative to the distal end of the single-use portion and said driven element at the proximal end of the single-use portion comprises first gear that mounts over the gear shaft in the assembled endoscope to be rotated thereby about an axis transverse to the cannula axis, a second gear engaging the first gear to be rotated thereby about an axis parallel to the cannula axis, a third gear affixed to the second gear to rotate therewith, and a fourth gear mating with the third gear to be rotated thereby about the cannula axis and thereby rotate the distal end of the cannula about the cannula axis relative to the proximal end of the single-use portion.

10. The compact robotic endoscope of claim 1, in which said distal end of the cannula is configured to angulate through said selected angle and said driven element at the proximal end of the single-use portion comprises a rotary disc that mounts over the gear shaft in the assembled endoscope to be rotated thereby about an axis transverse to the cannula axis and said single use portion comprises guide wires coupling the rotary disc to the distal end of the cannula such that rotation of the rotary disc causes the distal end of the cannula to angulate relative to the cannula axis.

11. A compact robotic endoscope set of a reusable portion and two different single-use portions a first of which has a motor-driven cannula rotation and a second has a motor-driven angulation of a distal end of a cannula, wherein:
the reusable portion is configured for being hand-held and has an elongated slot that has an open side extending along a cannula axis, has an internal motor and a gear shaft extending into said slot and configured to be selectively rotated by the motor about a shaft axis, and an electrical contact in said slot;
the first single-use portion is configured to be coupled with said reusable portion by being snapped into said slot solely in a direction transverse to the cannula axis to thereby form a first assembled endoscope, and comprises a distal end with an imaging module, a proximal end with a socket configured to fit over said gear shaft to be rotated thereby about the shaft axis in the first assembled endoscope and a gear set coupling the socket with the distal end of the first single-use portion to impart rotation thereof relative to the proximal end thereof about the cannula axis in a selected angular direction and to a selected degree;
the second single-use portion also is configured to be coupled with said reusable portion by being snapped into said slot solely in a direction transverse to the cannula axis to thereby form a second assembled endoscope, and comprises a distal end with an imaging module, a proximal end with a socket configured to fit over said gear shaft to be rotated thereby about the shaft axis in the second assembled endoscope and guide wires coupling the socket with the distal end of the second single-use portion to angulate said distal of the distal end of the second single-use portion relative to the proximal end thereof in a selected direction and to a selected degree relative to the cannula axis;
thereby enabling the same reusable portion to form the first endoscope in which the distal end of the first single-use portion robotically rotates or the second endoscope in which the distal end of the single-use portion robotically angulates.

12. The compact robotic endoscope set of a reusable portion and two different single-use portions of claim 11, in which the proximal end of the first single-use portion further comprises a manual control coupled with said distal end of the first single-use portion to impart angulation thereof relative to the proximal end of the first single-use portion in a selected direction and to a selected degree relative to the cannula axis.

13. The compact robotic endoscope set of a reusable portion and two different single-use portions of claim 11, in which the proximal end of the second single-use portion further comprises a manual control coupled with said distal end of the second single-use portion to impart rotation thereof relative to the proximal end of the second single-use portion in a selected direction and to a selected degree about the cannula axis.

14. The compact robotic endoscope set of a reusable portion and two different single-use portions of claim 11, in which the reusable portion furthest comprises a display mounted thereof and having a proximal face configured to display images acquired with said imaging module and a distal face with a light source and one or more forward-facing cameras (FFC) configured to image an object distal therefrom and provided with autofocus on said object as the endoscope moves relative to the object.

15. A robotic endoscope that is single use in its entirety and comprises:
a handle that has a distal end and a proximal end and is configured to be hand-grasped by a user and further has a working channel port at the proximal end thereof, an internal motor with a motor shaft that rotates when the motor is energized, and buttons configured to selectively energize the motor to rotate the motor shaft in a selected angular direction and through a selected angle;
a hub that has a proximal end permanently mounted to the distal end of the handle to rotate relative thereto about a cannula axis;
a cannula that has a proximal end permanently secured to the distal end of the hub, extends therefrom along the cannula axis, and has an imaging module and a working channel port at a distal end thereof;
a working channel with constant internal area, extending from the working channel port at the proximal end of the handle to the working channel port at the distal end of the cannula;
coupling elements connecting said motor shaft to the distal end of the cannula and configured to translate rotation of the motor shaft in a selected angular direction through a selected angle to angulation of the distal end of the cannula in a selected direction and to a selected degree relative to the cannula axis; and
a transmission facility coupled to said imaging module to transmit images acquired thereto to a display outside the single-use endoscope.

16. The robotic endoscope of claim 15, further comprising a remote display and control facility coupled with said transmission facility and configured to control said imaging module and to display images acquired with the imaging module.

17. The robotic endoscope of claim 16, in which said transmission facility is wireless.

18. An endoscope that is single use in its entirety and comprises:
a handle that has a distal end and a proximal end and is configured to be hand-grasped by a user and further has a working channel port at the proximal end thereof, and a lever configured to be manually moved between first and second positions relative to the handle;
a cannula that has a proximal end permanently secured to the distal end of the hub, extends therefrom along the cannula axis, and has an imaging module and a working channel port at a distal end thereof;
a working channel with constant internal area, extending from the working channel port at the proximal end of the handle to the working channel port at the distal end of the cannula;
a motion translating mechanism inside the handle that is coupled to the lever and to the distal end of the cannula and is configured to translate motion of the lever in a selected direction and to a selected degree to angulation of the distal end of the cannula in a selected direction and to a selected degree relative to the cannula axis; and
a transmission facility coupled to said imaging module to transmit images acquired thereto to a display outside the single-use endoscope.

19. The endoscope of claim 18, in which the lever is configured to pivot about an axis transverse to the cannula axis and the motion translation mechanism comprises an element coupled with the lever to rotate with pivoting of the lever and guide wires connecting the element to the distal end of the cannula.

20. The endoscope of claim 18, further comprising a remote display and control facility coupled with said transmission facility and configured to control said imaging module and to display images acquired with the imaging module.
